(19) United States
(12) Plant Patent Application Publication
(10) Pub. No.: US 2020/0113106 P1
(43) Pub. Date: Apr. 9, 2020

(54) Calla lily plant named 'Royal Plum'

(50) ;

(71) Applicant: Dummen Group B.V., De Lier (NL)

(72) Inventor: Peter Beckman, Santa Cruz, CA (US)

(21) Appl. No.: 16/602,422

(22) Filed: Oct. 1, 2019

Related U.S. Application Data
(60) Provisional application No. 62/766,205, filed on Oct. 4, 2018.

(51) Int. Cl. A01H 6/56

(52) U.S. Cl. PLT/490

(57) ABSTRACT

‘Royal Plum’ is a new variety of calla lily plant having red-purple colored spathes. The flower stems are strong and long lasting. The leaves are green with maculations present.

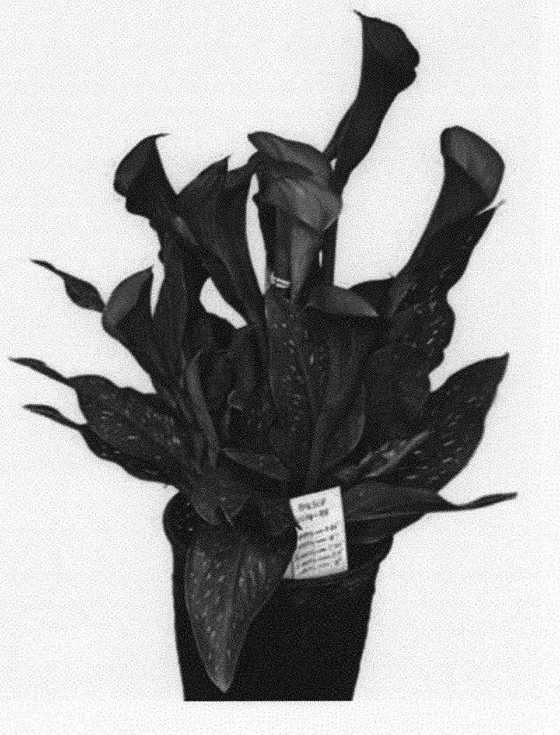

### CROSS REFERENCE TO RELATED APPLICATION

This application claims the benefit of U.S. Provisional Patent Application Ser. No. 62/766,205, having a filing date of Oct. 4, 2018, and entitled “Varieties of Z. sprengeri Calla Lily Plants”, the entire disclosure of which is incorporated herein by reference.

Botanical classification: Zantedeschia sprengeri.

Varietal denomination: ‘Royal Plum’.

### BACKGROUND OF THE NEW PLANT

The present invention comprises a new and distinct cultivar of calla lily, botanically known as Zantedeschia sprengeri and hereinafter referred to by the cultivar name ‘Royal Plum’.

‘Royal Plum’ is a product of a planned breeding program and the new cultivar was selected from the crossing of unpatented Zantedeschia rehmannii x Zantedeschia elliottiana seedling 52059A (female parent) with unpatented Zantedeschia rehmannii x Zantedeschia elliottiana seedling 52059A (male parent). ‘Royal Plum’ was selected in Santa Cruz, Calif. as one flowering plant within the progeny of the stated cross.

Asexual reproductions of ‘Royal Plum’ have demonstrated that the combination of characteristics as herein disclosed for the new variety are retained and reproduced true to type through successive generations of asexual reproduction.

Color references are made to the 2001 R.H.S. Colour Chart of The Royal Horticultural Society of London, 4^{th} Edition, except where general color terms of ordinary significance are used.

The present invention has not been evaluated under all possible environmental conditions. The phenotype may vary with variations in environment without a change in the genotype of the plant.

The following traits have been repeatedly observed and determined to be basic characteristics of ‘Royal Plum’ which, in combination, distinguish this calla lily as a new and distinct cultivar:

1. Red-purple colored spathes;

2. Strong peduncles; and

3. Leaf maculations present.

Table 1 provides a comparison of ‘Royal Plum’ to similar Zantedeschia sprengeri varieties ‘Twilight’ (unpatented) and ‘California Red’ (unpatented). Further, the plant and spathes are taller than those of ‘Twilight’.

TABLE 1

| Comparison | | Comparison | Present variety |
|---|---|---|---|
| variety | Differing trait(s) | expression | expression |
| ‘Twilight’ | Spathe color | Lavender | Red-Purple Group |
| | | | RHS 61A |
| | Peduncle strength | 14 days to | 28 days to nodding |
| | | nodding | |
| ‘California Red’ | Leaf maculations | None present | Present |

### BRIEF DESCRIPTION OF THE DRAWING

The accompanying photographic drawing illustrates the new variety, with the colors being as nearly true as is possible with color illustrations of this type.

### DESCRIPTION OF THE NEW PLANT

Type: Deciduous.
Main use: Potted plant and cut flower.
Disease/pest resistance: Strong tolerance to Pythium root rot and Erwinia bulb soft rot.
Leaves: Green, with maculations present.
Spathe:
   Length.—Medium.
   Width.—Medium.
   Throat spot.—Present.
   Color.—Red-Purple Group RHS 61A.
Peduncle: Strong and erect, with no nodding observed until day 28.

I claim:

1. A new and distinct variety of calla lily plant named ‘Royal Plum’ as herein described and illustrated.
